(19) United States
(12) Patent Application Publication
(10) Pub. No.: US 2020/0365274 A1
(43) Pub. Date: Nov. 19, 2020

(54) HEALTHCARE DATA INGESTION AND ANALYSIS SYSTEM AND METHOD

(71) Applicant: Accumen, Inc., San Diego, CA (US)

(72) Inventors: Cindi Karl, San Diego, CA (US); Clay Tyler, San Diego, CA (US); Joseph Jager, San Diego, CA (US); Bobby Shreckengost, San Diego, CA (US); Bret Awbrey, San Diego, CA (US); Dan Smoragiewicz, San Diego, CA (US); Christopher Patti, San Diego, CA (US)

(21) Appl. No.: 16/568,750

(22) Filed: Sep. 12, 2019

Related U.S. Application Data
(63) Continuation-in-part of application No. 15/424,396, filed on Feb. 3, 2017.

(51) Int. Cl. G16H 50/30; G16H 10/60; G16H 10/40; G16H 15/00

(52) U.S. Cl. CPC G16H 50/30; G16H 15/00; G16H 10/40; G16H 10/60

(57) ABSTRACT

A healthcare data ingesting and display system and method may ingest healthcare data from a plurality of healthcare data sources wherein each healthcare data source has its own data format. The system and method may ingest the disparate data, conform the data from the disparate sources and generate dashboards based on the data. In certain embodiments, the system and method may be used for lab testing data, imaging data, patient blood management data or other healthcare operations' performance data.

### PRIORITY CLAIMS/RELATED APPLICATIONS

This application is a continuation in part and claims priority under 35 USC 120 to U.S. patent application Ser. No. 15/424,396, filed Feb. 3, 2017 and entitled “Laboratory Data Ingestion and Analysis System and Method”, the entirety of which is incorporated herein by reference.

### FIELD

The disclosure relates generally to a system and method for ingesting and displaying various types of healthcare data including, without limitation, laboratory, imaging, and operations' performance data (collectively “healthcare data”).

### BACKGROUND

Clinical laboratories are complex, require sophisticated management, and produce extensive data. There are some software systems available, but most are extremely limited resources.

Typically, software systems that are used in connection with laboratory management and its workflow, etc. are systems that are limited in scope to a single laboratory/data source and often only are able to manage and provide limited insights with respect to that single laboratory/data source. In some cases, those typical systems are a laboratory data storage system from which it is difficult to generate insights. As a result, these systems are unable to provide insights that are based on a wider scope of data sources. These existing lab management systems may monitor and manage lab performance and testing performance of the specific lab for which they are built, but cannot be used for a wider scope of data sources.

Other existing systems may monitor and manage patient blood within the lab or hospital (separately from the lab management systems). These patient blood management and monitoring systems are limited and do not effectively monitor usage of blood for patients (very often being a factor in more blood than is recommended given the circumstances being utilized). Furthermore, these existing systems do a relatively poor job of ensuring transfusion safety in that these system do not do an adequate job of preventing adverse events due to a transfusion. These systems also contribute to blood wastage and blood outdating.

Laboratory data, imaging data, performance data and patient blood data are several different types of healthcare data. It is desirable to be able to ingest this healthcare data and provide various analysis of the healthcare data.

### BRIEF DESCRIPTION OF THE DRAWINGS

FIG. 1 illustrates an example of a healthcare data ingestion and analysis system;

FIG. 2 illustrates a method for healthcare data ingestion and analysis that may be performed by the healthcare data ingestion and analysis system;

FIG. 3 illustrates an example of a scorecard for a lab performance that may be generated by the laboratory data ingestion and analysis system;

FIG. 4 illustrates an example of a dashboard that may be generated by the laboratory data ingestion and analysis system;

FIG. 5 illustrates an example of a mini-card that may be generated by the laboratory data ingestion and analysis system;

FIGS. 6A and 6B illustrate further details of a line graph that is part of the dashboard shown in FIG. 4;

FIGS. 7A and 7B illustrate further details of a window with tabular data about a particular data point of the line graph;

FIGS. 8 and 9 are examples of the graphs that may be generated by the laboratory data ingestion and analysis system;

FIGS. 10A and 10B illustrate an example of a control chart that may be generated by the laboratory data ingestion and analysis system;

FIG. 11 illustrates an example of a chart generated by clicking on an area in the mini-chart of the dashboard;

FIGS. 12A and 12B illustrate an example of a global filter user interface for the laboratory data ingestion and analysis system;

FIG. 13 illustrates an example of an advanced filter user interface for the laboratory data ingestion and analysis system;

FIG. 14 illustrates an example of a blood analytics dashboard generated by the system;

FIG. 15 illustrates an example of a red cell metric dashboard generated by the system;

FIG. 16 illustrates an example of a red cell metric provider detail dashboard generated by the system;

FIG. 17 illustrates an example of a patient blood management monthly operating review screen generated by the system;

FIG. 18 illustrates an example of a patient blood management percent single unit transfusion trend view generated by the system;

FIG. 19 illustrates an example of a patient blood management percent single unit transfusion hospital print view generated by the system;

FIG. 20 illustrates an example of a patient blood management percent single unit transfusion specialty print view generated by the system;

FIG. 21 illustrates an example of a patient blood management pre-transfusion trend print view generated by the system;

FIG. 22 illustrates an example of a patient blood management pre-transfusion hemoglobin hospital print view generated by the system; and

FIG. 23 illustrates an example of a patient blood management pre-transfusion hemoglobin specialty print view generated by the system.

### DETAILED DESCRIPTION OF ONE OR MORE EMBODIMENTS

The system and method described below is particularly applicable to a healthcare data ingestion and display system and method that may be used for healthcare data, such as lab performance data or patient blood management data or imaging data, and it is in this context that the disclosure will be described. It will be appreciated, however, that the healthcare data ingestion and display system and method has greater utility since the system may be used for other types of healthcare data; the system and method may be implemented using different computer-based technology or architecture than that disclosed below and modifications may be made to the disclosed system and method without departing from the scope of this disclosure. In addition, the system and method may be used to generate insightful visuals of key performance metrics and indicators for stakeholders in various healthcare related entities, such as hospitals, health systems and independent laboratories, to drive efficiency and monitor outcomes of performance (including productivity, quality, service, and cost) and process improvement.

In the context of patient blood management, the system and method described below may improve transfusion safety using a multi-modal strategy focused on providing physicians with the data necessary to help avoid adverse events, and recognize and report transfusion reactions. The system and method also delivers optimized inventory management to provide physicians with the data and forums necessary to establish monitoring and reporting processes and to ultimately endeavor to reduce blood product wastage and outdate s. The system and method may further provide:
Change Management tools and resources to bolster physician engagement in implementing and sustaining blood management and utilization programs.
Patient Blood Management program resources designed to standardize systems, processes, and practices across disciplines and hospitals within a health system (all under the direction of a Medical Director).
Enabled Blood-centric Technology Suite seamlessly integrated into existing systems to drive actions and improvements in operations.
A tool for continuous Monitoring and Oversight by Medical Professionals of clinical, regulatory, and blood safety guidelines and processes.
A means to exchange data between blood providers and Health Systems for use in Blood Product Management.

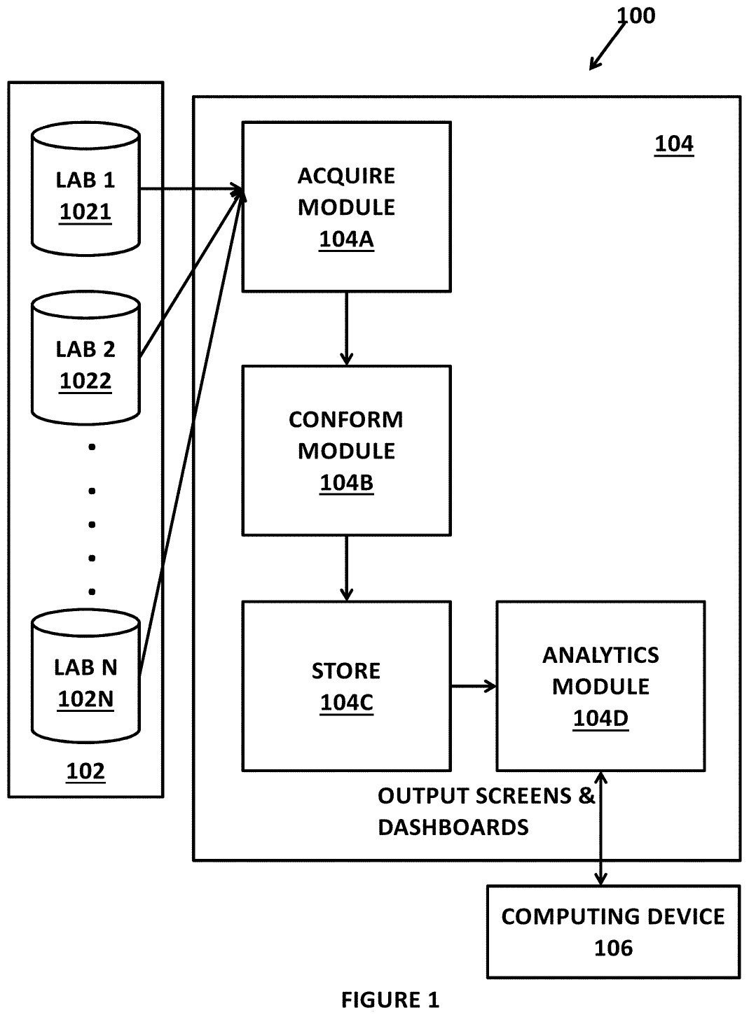

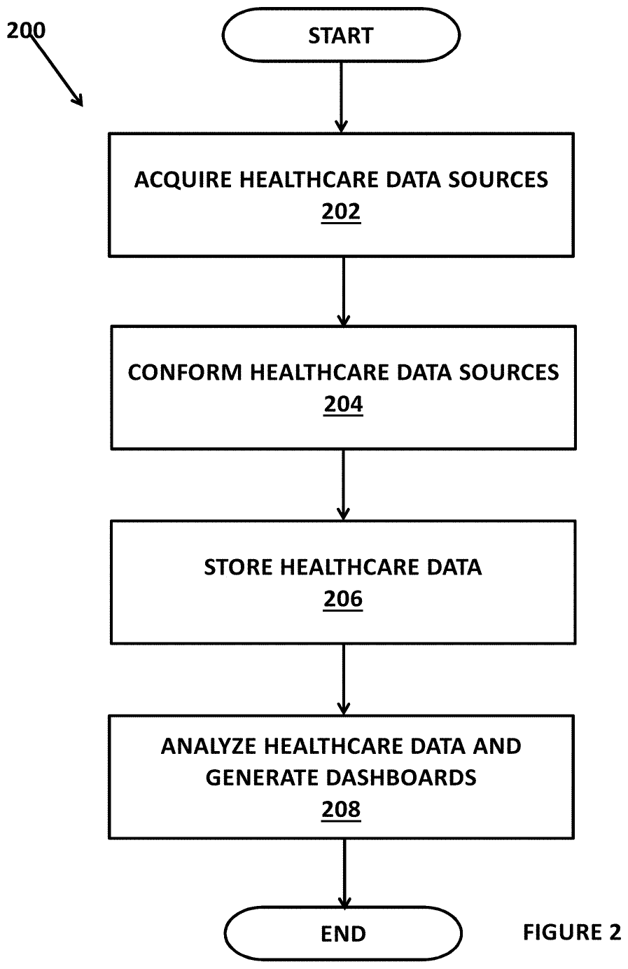

FIG. 1 illustrates an example of a healthcare data ingestion and analysis system 100 that may be used for ingesting and analyzing both lab test and performance data for laboratory excellence or patient blood management laboratory data or imaging data, and FIG. 2 illustrates a method 200 for healthcare data ingestion and analysis that may be performed by the healthcare data ingestion and analysis system. The system 100 may have one or more healthcare data sources 102 that may be ingested into the system. The one or more healthcare data sources may be part of the system 100 or may be remote from the system 100 and accessed over a communications path, such as the Internet, Ethernet, a wireless network, a computer network and the like. In one embodiment, each of the one or more healthcare data sources may be a laboratory data source, such as lab 1 data source 1021, lab 2 data source 1022, . . . , lab N source 102N or an imaging data source (that may also be stored in each laboratory data source or elsewhere), wherein each healthcare data source may contain healthcare data about the laboratory, its tests, imaging data, other performance data, etc. in its own proprietary data format. For example, the healthcare data sources may use various data formats including the well-known HL7 data format or a flat file format or other known health data formats. In addition to the healthcare data systems shown in FIG. 1, the system may also be used to ingest, normalize, and generate dashboards from financial information systems. The data sources ingested by the system may include primary data sets including finance, accounting, human resources, sales and system log data from each laboratory, and reference data sets (including employees and locations for each laboratory). The primary data sets may further include imaging data, lab order data, lab results data, labor data, blood orders data, blood inventory data, pharmacy data, encounters data, outcomes data, procedures data, outreach data, supply chain data, and billed volume data.

The system 100 may further comprise a backend component/platform 104 that is coupled to the one or more healthcare data sources 102 over the communications path. The backend component/platform 104 (and each module/component of the backend 104) may be implemented in software, hardware, or a combination of hardware and software. For example, the backend component/platform 104 may be implemented using one or more computing resources, such as server computers, database server computers, application server computers, blade servers, data center computing resources, cloud based computing resources that include one or more processors and memory. The modules 104A, 104B and 104D may be implemented using a plurality of lines of instructions/computer code that may be stored in the memory and executed by a processor of the backend component 104 to implement the operations and functions of the backend component as described below. Each of the modules 104A, 104B and 104D may be alternatively implemented in hardware and each module may be a hardware device that performs the operations and functions of each module as described below. A store 104C of the backend component 104 may be storage for system data, business logic that is part of the conform module 104B that is used to process the incoming healthcare data, the logic used by the analytics module that performs the analysis of the healthcare data and generates the outputs screens and the dashboards, the incoming healthcare data and the conformed healthcare data stored by the system. The store 104C may be implemented using hardware (such as a database server, a hardware data storage system, a storage device) or software (such as a software database system or a software based data storage system).

The acquire module 104A may include an interface that receives/acquires the data from each of the healthcare data sources (process 202 in FIG. 2). The acquire module may include, for example, an application programming interface (“API”) that allows the acquire module to gather the information from each of the healthcare data sources. Alternatively, the acquire module may have other mechanisms (such as to receive batched healthcare data or other forms of the data) to gather/poll each healthcare data source and ingest the healthcare data for that particular healthcare data source (with its possibly unique healthcare data format) into the system for processing and analysis. The acquire module 104A may be a single acquire module that receives the data from each of the data sources, or—alternatively—the acquire module 104A may be an acquire module 104A for each different data source wherein each acquire module 104A may be optimized to receive and process the incoming data from the particular data source.

As new healthcare data sources are going to be ingested into the system, the acquire module 104A may be supplemented with new APIs or other mechanism to be able to retrieve the healthcare data for the new healthcare data source with its own unique data format. The acquire module 104A may also translate the data from each data source into a common data format used by the store 104C of the system. The acquire module 104A may also acquire a direct copy of the source data in tabular format. In some embodiments, the system may have separate database for the storage of the data from each data source. The acquire module 104A may also process data in the well-known format into a common format, such as by using a commercially available tool from Carista (www.caristaapp.com). The acquire module 104A may also process data in the HL7 data format (ingesting and mapping) such as by using a well-known commercially available tool called Iguana (www.interfaceware.com/iguana.html). The acquire module 104A may use SSIS to ingest the data from the data sources. SSIS is a commercially-available platform for data integration and workflow applications that features a fast and flexible data warehousing tool used for data extraction, transformation, and loading (ETL). The tool may also be used to automate maintenance of SQL Server databases and updates to multidimensional cube data. The acquire module 104A may also, during the ingestion process, generate audit columns. The output of the acquire module is a set of data for each healthcare data source in that healthcare data sources data format.

Once the healthcare data for each respective data source is retrieved and ingested by the acquire module, the healthcare data for each such data source may be fed into the conform module 104B. The conform module 104B may include a plurality of business logic and rules that may be used to process each piece of ingested healthcare data. The conform module, using the business logic, may conform the healthcare data from the disparate sources and normalize the ingested healthcare data to the internal data structure of the system 100 (process 204 in FIG. 2). The conform module 104B may be a single conform module or alternatively the conform module 104B may be conform module 104B for each different data source. The conform module 104B may cleanse and normalize the source data using the business logic rules and generate the audit data for each data source. The conform module 104B may also add meta data to align certain types of data in the various data sources and may generate/add hash keys for secure data.

As a result of this module, normalized healthcare data may be stored in the store 104C (process 206 in FIG. 2). The normalized healthcare data is important so that the system is able to process and analyze that healthcare data and generate analysis of that healthcare data as well as the healthcare data from each of the healthcare data sources. The store 104C may store the data from the various data sources in one or more databases/datamarts in a target data schema. In the system, there may be data schemas for the conform module that map the data from each data source (using a plurality of lines of computer code/script) into the common database used by the system. In the common database, the different types of data from each data source may be normalized so that the analytics may be generated as described below.

The analytics module 104D may retrieve various healthcare data from the store 104C and generate output screens and dashboards based on the various pieces of healthcare data (process 208 in FIG. 2). In one embodiment, the system 100 may be used to generate laboratory performance data dashboards and reports based on the pieces of healthcare data as described below in more detail. In another embodiment of the system 100, the system may be used to generate blood patient management data dashboards and reports based on the pieces of healthcare data as described below in more detail. In accordance with this disclosure, the system 100 may be used to generate various other healthcare related dashboards and reports and those other healthcare related dashboards and reports are within the scope of this disclosure. The analytics module 104D may generate the dashboards using a plurality of business logic rules wherein each business logic rule includes a formula for calculating a metric that is a standard for measuring an evaluating data. For example, in the laboratory example, the metrics may include high priority test (STAT) turn-around time (TAT), In-Patient Morning Result (IPMR), emergency department (ED) test TAT, as soon as possible (AP) test TAT, unacceptable specimens, and corrected reports. An exemplary relatively simple business logic rule may be a numerator, such as a count of designated tests within a target time, over a denominator, such as total count of designated tests.

In the laboratory example described below, various analytics/metrics described below may be generated including, for example, high priority test (STAT) turn-around time (TAT), In-Patient Morning Result (IPMR), emergency department (ED) test TAT, as soon as possible (AP) test TAT. In addition, for patient blood data or imaging data, different metrics may be programmatically generated by the system. For the imaging data and metrics/analytics, the analytics/metrics are similar to the lab metrics and may include imaging spend based on volume, labor and productivity as well as turnaround time (TAT) performance for imaging.

Each of the different metrics has a plurality of business logic and rules which generate the analytical data based on the data from the data sources. For example, for the ED TAT metric, the analytics (with each analytic having a business rule/logic associated with the analytic) may include a collect specimen to completed test result (collect to result) analytic (that may be in the form of a percentage chart, a histogram or a control chart), an in-lab to completed test result (in-lab to result) analytic (that may be in the form of a percentage chart, a histogram or a control chart), a collect to in-lab analytic (that may be in the form of a percentage chart, a histogram or a control chart), a volume of test analytic (that may be in the form of a column or a combo chart), an exceptions count analytic, an ER phlebotomist (Phleb) Productivity analytic in column form that may be view by hour, by facility and Phleb, an overall Lab ED TAT analytic that may be a multiline Chart, an ED Phleb Exceptions analytic that may be in column format, an order to result analytic (that may be in the form of a percentage chart, a histogram or a control chart) and an order to collect analytic (that may be in the form of a percentage chart, a histogram or a control chart).

For example, for the order to collect analytic may have its own unique business rules and logic to generate that analytic. For purposes of determining this analytic, the numerator of the rule may be a total time from collection to receipt (as obtained from the data from the data sources) and the denominator may be count of the eligible ID tests for the particular lab for which the analytic is being generated. The business logic specifies that the target for this analytic is 15 minutes. Thus, using this programmed business rule and logic, the system is able to generate this analytic from the data from the data sources for a particular lab. As another example, the order to result analytic may have its own unique business rules and logic to generate that analytic. For purposes of determining this analytic, the numerator of the rule may be a Total time from Order to Result and the denominator may be count of eligible ED tests for the particular lab. The business logic/rule may also specify that the target for this analytic is less than 60 minutes. Similarly, each analytic may include a unique business rule/logic that is used to generate the analytics generated by the system.

The output screens and dashboards generated by the system may be downloaded and displayed on one or more computing devices 106 that can access the backend 104 over a communications path such as a computer network, the Internet, Ethernet, a wireless network and the like. In one implementation, each computing device may execute a well-known browser application and communicate/exchange data with the backend 104 using a known HTML protocol and the backend 104 may have a web server that generates the HTML code that may be sent to each computing device. However, the system may also be implemented as cloud based services and then the computing device will access the backend 104 in a different manner that is known in the art. Thus, the system is not limited to any particular way in which the backend 104 and the computing device 106 interact with each other. Each computing device 106 may be a processor-based device with memory, persistent storage, a display and wireless or wired communications circuits. For example, each computing device 106 may be a smartphone device, such as an Apple iPhone or Android operating system based device, a personal computer, a tablet computer, a laptop computer and the like.

The system 100 is addressing a technical problem of trying to analyze voluminous healthcare data from disparate data sources that have different data formats. The technical solution to that problem is to use the plurality of business logic rules of the conform module 104B to normalize the healthcare data from the disparate healthcare data sources so that the analytics module 104D is able to process the healthcare data and perform the analytics to generate the various output screens and various dashboards for the various different healthcare data from the various healthcare data sources.

Lab Performance Data Analysis and Data Visualization Examples

To illustrate the power and capabilities of the laboratory data ingestion and analysis system, examples of the data analytics generated by the system using the laboratory data for lab test performance is described in more detail.

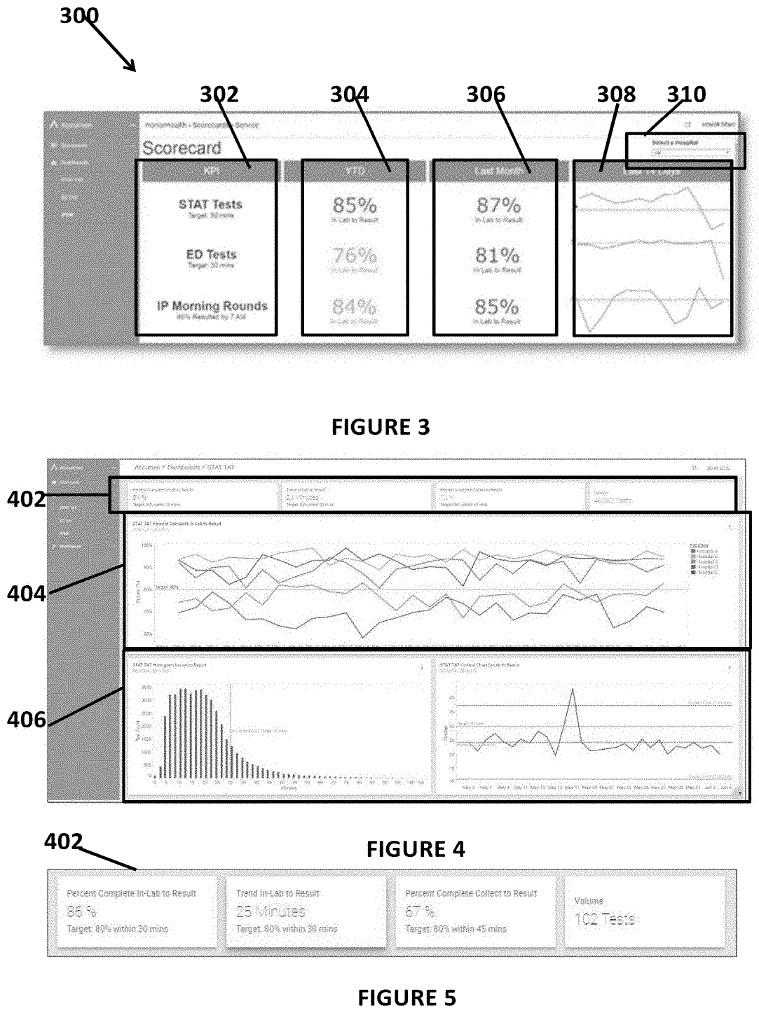

FIG. 3 illustrates an example of a scorecard 300 for lab performance that may be generated by the laboratory data ingestion and analysis system 100 shown in FIG. 1. In one implementation, the scorecard 300 may be a landing page when an authorized user logs into the laboratory data ingestion and analysis system 100. The scorecard may display, for each user of the system, the resultant metrics (STAT TAT, ED TAT, IPMR) for the lab owned or being managed by the authorized user. Additional metrics may be added as they flow through the metric deployment process and may be accessed via a navigation bar. As shown in FIG. 3, the scorecard may include a key performance indicator (KPI) portion 302 that lists each metric, a year to date (YTD) portion 304 that shows the YTD percentage for each KPI for the particular lab facility being reviewed, a last month portion 306 that shows the percentage for each KPI for the particular lab facility being reviewed, a chart portion 308 showing a chart for each KPI over the last pre-determined period of time (e.g., 14 days), and a select facility portion 310 that allows the user to change facilities/sites (for any facilities whose data the user is authorized to access), and regenerate the scorecard with the KPIs for the selected facility wherein the selected facility/site may be (for example, a specified hospital). In the scorecard, each KPI may have text below the KPI name indicating a target value for the particular KPI. In the scorecard, the color of the text for the YTD portion 304 and the last month portion 306 may change depending on the value indicated by the text (the performance on that metric. For example, the text may be green if the facility/site achieved the target for the particular KPI, the text may be yellow if the facility/site is within 15% of the target for the KPI and the text may be red if the facility/site does not meet the target. The scorecard may provide users with a high-level easy-to-read overview of system or individual facility performance. Color-coding the results provides an immediate visual about which metrics the organization is achieving or falling short of performance expectations in any area. The user will accordingly know which areas require additional attention, and the user can use the dashboard to drill into more detail to further investigate issues and conduct root-cause analyses. From the scorecard shown in FIG. 3, the system may provide the user with a navigation menu that allows the user to navigate to the various performance results of the lab or labs that are owned/controlled/managed by the particular user.

FIG. 4 illustrates an example of a dashboard that may be generated by the laboratory data ingestion and analysis system. The dashboard is a collection of content modules (e.g., charts) which tell a story about the lab data for a particular client. The dashboard is as real time as the data feed and can be filtered on a number of dimensions (date, facility, test code, etc.) as described below. The dashboard generally consists several separate areas called content modules that can contain charts, graphs, data grids, video content, etc. that may be relevant to the user. The benefits of the dashboards are that they provide views of lab performance across the system and facilitate root cause analysis by allowing the user to interact (roll around) with the data to better analyze and assess root cause to process variances or meaningful fluctuations in volume. For example, each dashboard may include a mini-chart portion 402, a line graph portion 404 and a data presentation portion 406 that may be displayed in single user interface. The portions shown in FIG. 4 are live data meaning that the user can move over any of the graphs, etc. to see more detailed data and may also select any of the graphs, etc. to see more detailed data.

FIG. 5 illustrates an example of a mini-card 402 that may be generated by the laboratory data ingestion and analysis system. The mini-card may provide two functions. First, the mini-card provides quick information related to the content of the dashboard, in this case of measure of the STAT TAT metric. Second, the mini-card acts as navigation buttons to more detailed content. Note the blue highlight on the second mini-card which indicates the active dashboard below it. The system may generate a different dashboard and mini-card for each lab metric of the user and its facilities/sites.

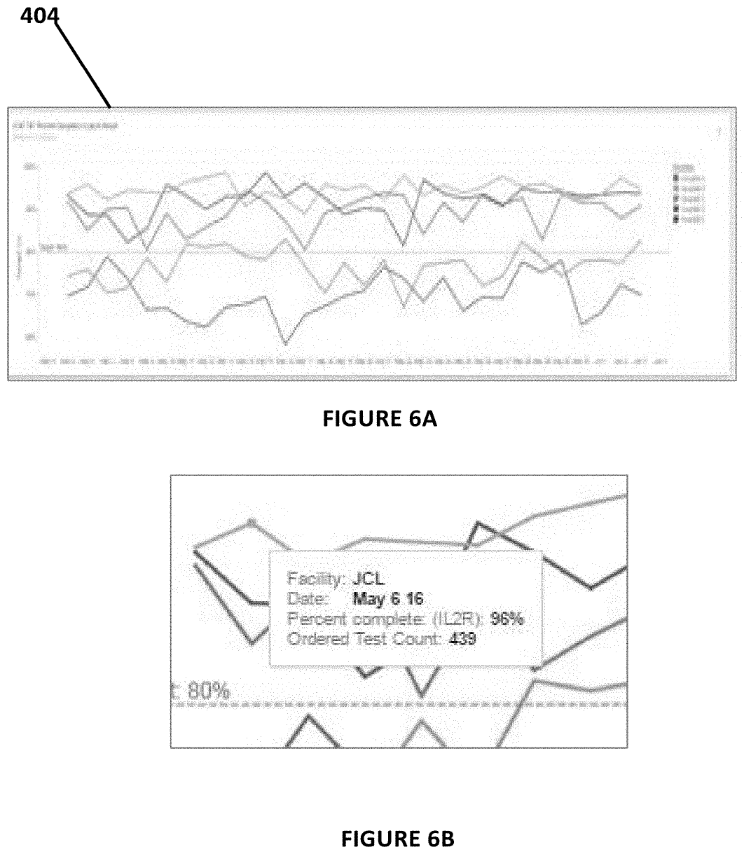

FIGS. 6A and 6B illustrate further details of a line graph 404 that is part of the dashboard shown in FIG. 4. The line graph defaults to show all facilities in the system each as a line in the chart. The line chart shows relative performance for the metric over the past 30 days. Clicking on a facility in the legend will toggle on/off its respective line in the chart. Clicking it again will redisplay it. FIG. 6B shows an example of the user interface when the user hovers over a data point to generate a tooltip user interface that shows relevant information about that data point. In the example in FIG. 6B, the tooltip user interface shows a hospital facility, a date, a performance against target, and a volume for the metric for the particular facility. In this manner, the user is able to quickly dive deeper into any of the data points on the chart.

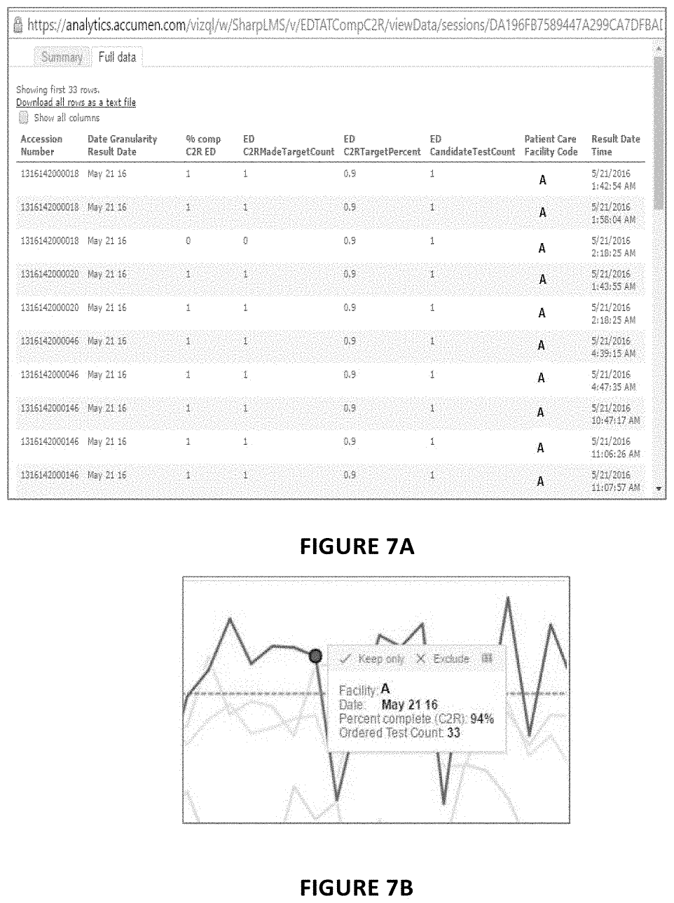

FIGS. 7A and 7B illustrate further details of a window with tabular data about a data point of the line graph and demonstrates how a user can use these views to conduct root-cause analysis. For example, the user may click on any data point and see a user interface such as shown in FIG. 7B. Note the header portion of the box where the user can temporarily “Keep only” or “Exclude” the data point on the chart. If you select either option, it will not refresh other dashboard data information since it is only manipulating the chart view. The user may also click on the tabular icon to the far right of the tool tip header in FIG. 7B and that action will open a window (example of which is shown in FIG. 7A) to the data associated with the data point. In the window in FIG. 7A, the user may click on the “Full data” tab. In this window, the accession numbers in the first column reference back to the lab information system (LIS).

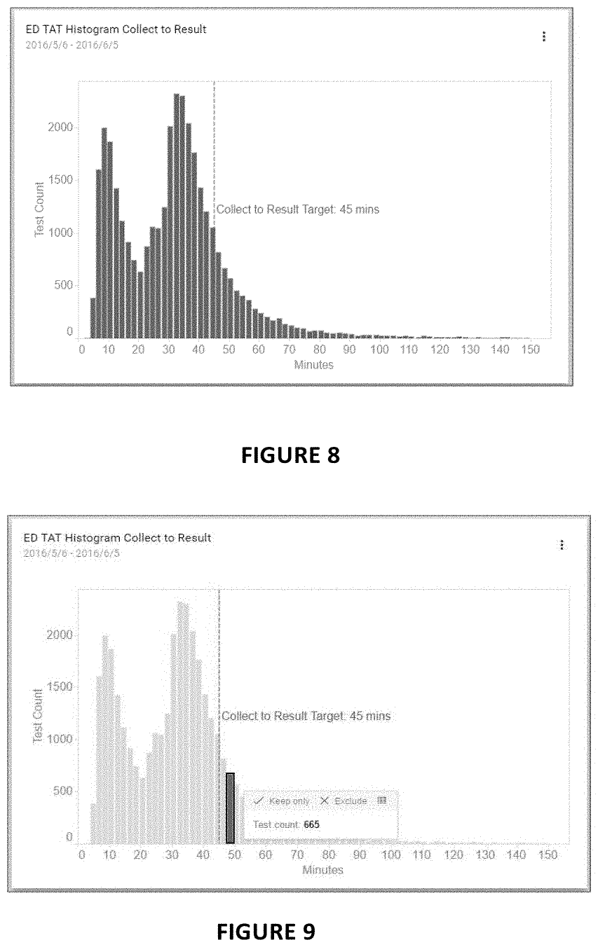

FIGS. 8 and 9 are examples of the graphs that may be generated by the laboratory data ingestion and analysis system. FIG. 8 shows an example of a Paretto chart and the chart, in this example, shows the number of tests (y-axis) resulted within N minutes (x-axis). There is a target to collect and result all tests within 45 minutes. Click on a column bar to isolate it and see the more information, in this case, test count volume is 665 tests resulted within 50 minutes of collection as shown in FIG. 9.

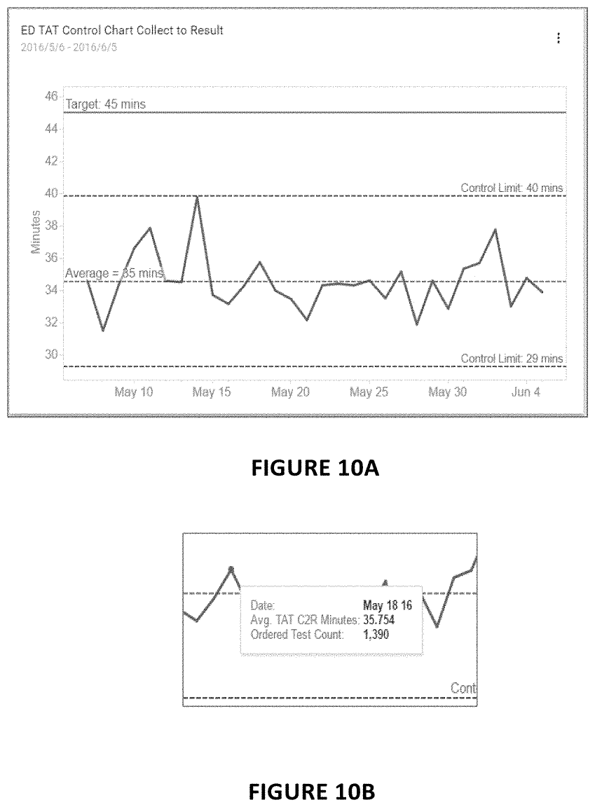

FIGS. 10A and 10B illustrate an example of a control chart that may be generated by the laboratory data ingestion and analysis system. The control chart may appear in a lower right portion of the dashboard in the data display portion 406 of the dashboard. A control chart shows process variability and here shows the amount of time in minutes (y-axis) to collect-to-result tests over time (x-axis) as shown in FIG. 10A. Note the target line (red) of 45 minutes. In this example, the hospital system as a unit is performing well within the target. The user may also hover you mouse pointer over a data point (example of which is shown in FIG. 10B) to see more detailed information. In this case the date, average time and test volume are shown for the data point of the chart.

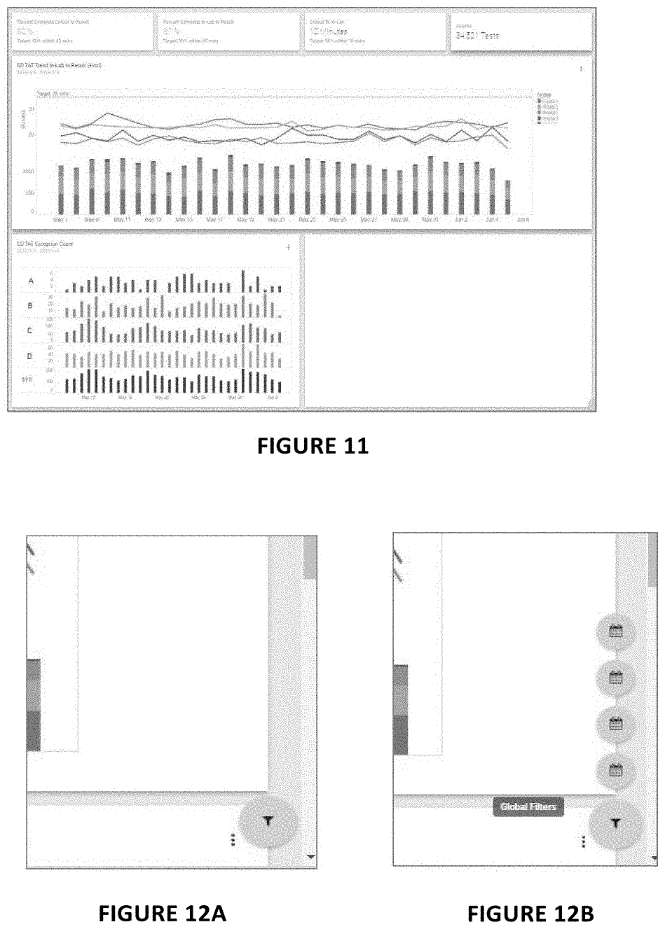

FIG. 11 illustrates an example of a chart generated by clicking on an area in the mini-chart of the dashboard. This chart may be generated when the user clicks on the right-most number of the mini-card of the dashboard. The charts (shown in FIG. 11) show test volume related to this turn-around-time metric. This view has two charts. The top chart is a combination line and stacked column charts. The line chart shows turnaround time for each facility with the stacked columns showing volume of each facility stacked to show system volume. The lower chart shows “missed” test counts. This show the volume of tests that didn't make target for each facility with the lowest showing the system.

FIGS. 12A and 12B illustrate an example of a global filter user interface for the laboratory data ingestion and analysis system. A default view for all dashboards shows performance for the last 30 days. Global filters provide quick and easy way to change date filters to apply to all dashboards. The global filters user interface may be accessed by hovering a mouse point over a yellow ball in a lower right-hand corner of a browser window as shown in FIG. 12A. When the user hovers over the ball, one or more balls appear as shown in FIG. 12B and each other ball has a preset date range that can be applied just by clicking on it. For example, the balls may have the following preset date ranges:

a. Last eight (8) complete quarters

b. Last twelve (12) complete months

c. Last thirty (30) days

d. Last twenty-four (24) hours

When a user clicks on one of the balls, the charts in the dashboard may be adjusted based on the selected date range. Thus, the global filters provide a quick and easy way to view data over different time periods and they persist while navigating to other dashboards.

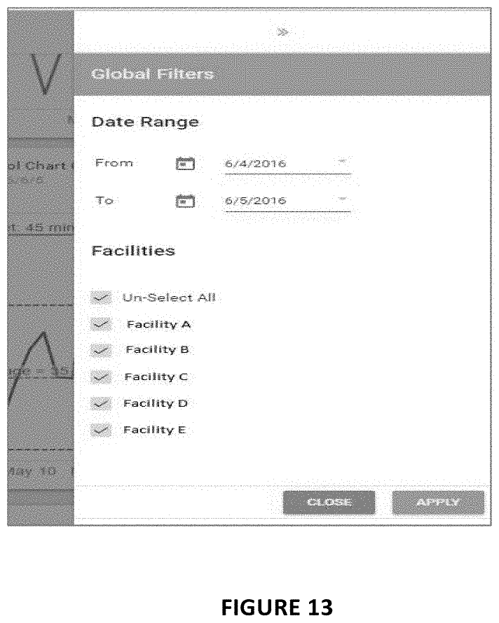

FIG. 13 illustrates an example of an advanced filter user interface for the laboratory data ingestion and analysis system. The advanced filters provide a means to apply custom filters to view and analyze the data as shown in FIG. 13.

Patient Blood Monitoring Data Analysis and Data Visualization Examples

To further illustrate the power and capabilities of the laboratory data ingestion and analysis system, examples of the data analytics generated by the system using the healthcare data for patient blood monitoring is described in more detail.

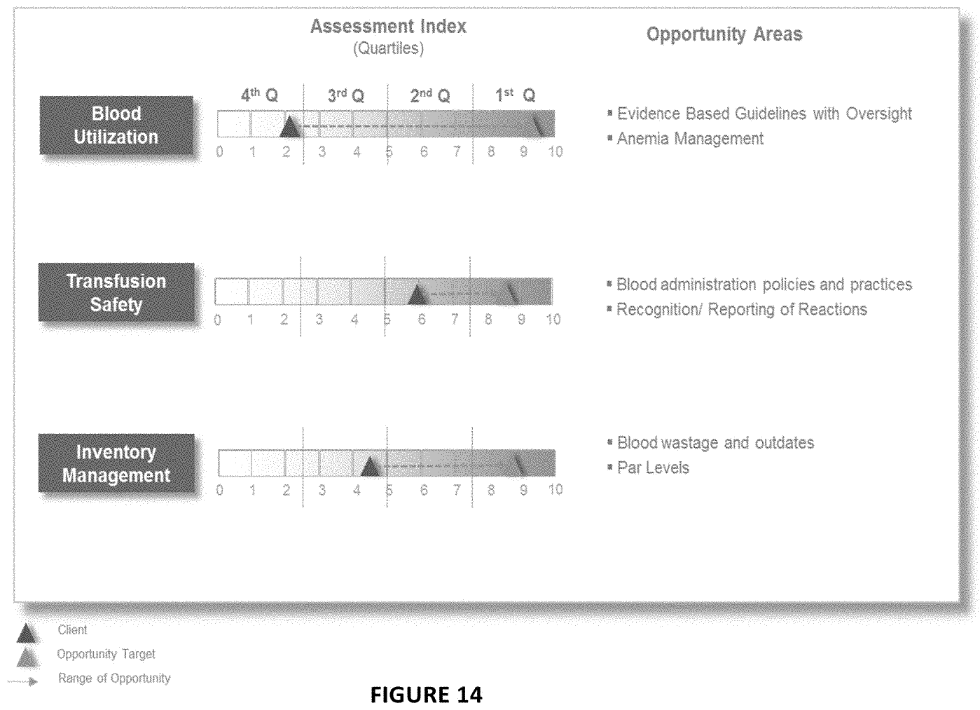

FIG. 14 illustrates an example of a blood analytics dashboard generated by the system. The analytic dashboard may show (generated from the data for the data sources) three dimensions of blood management that may include blood utilization, transfusion safety, and inventory management. For each of the dimensions, the dashboard may display an assessment index. The assessment index may include a current state for a particular laboratory as shown by the filled in triangle that sits along the assessment index. Each assessment index may be individually graded with numbers to show the range of values for each dimension. Each dimension thus shows as a line on the assessment index for the dimension an opportunity target that is the system's assessment of its ability to achieve greater blood management more in-line with its own designated goals (based on current state, culture, talent, technology, etc.).

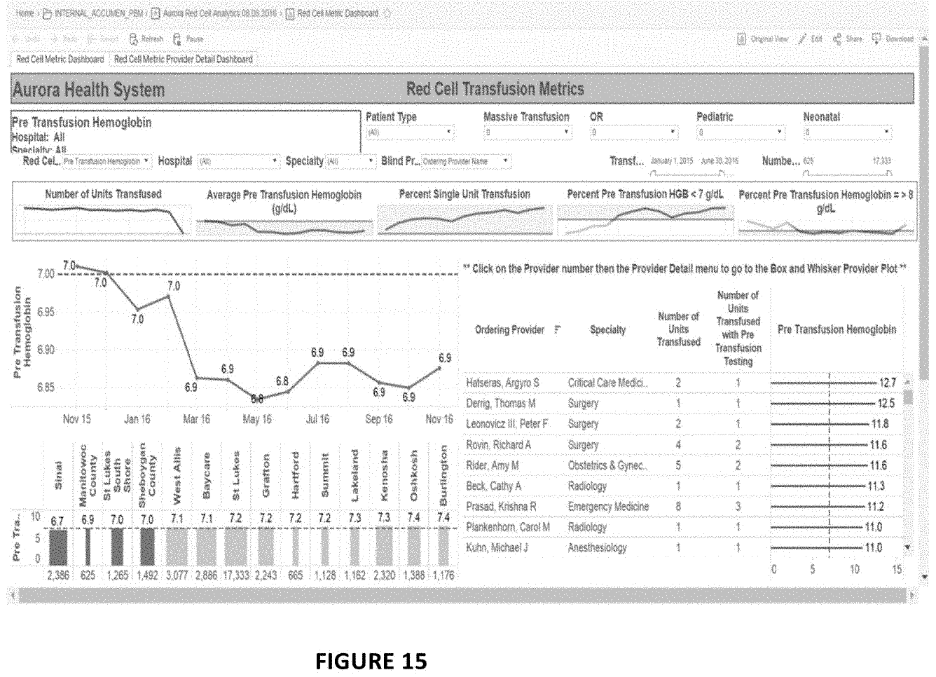

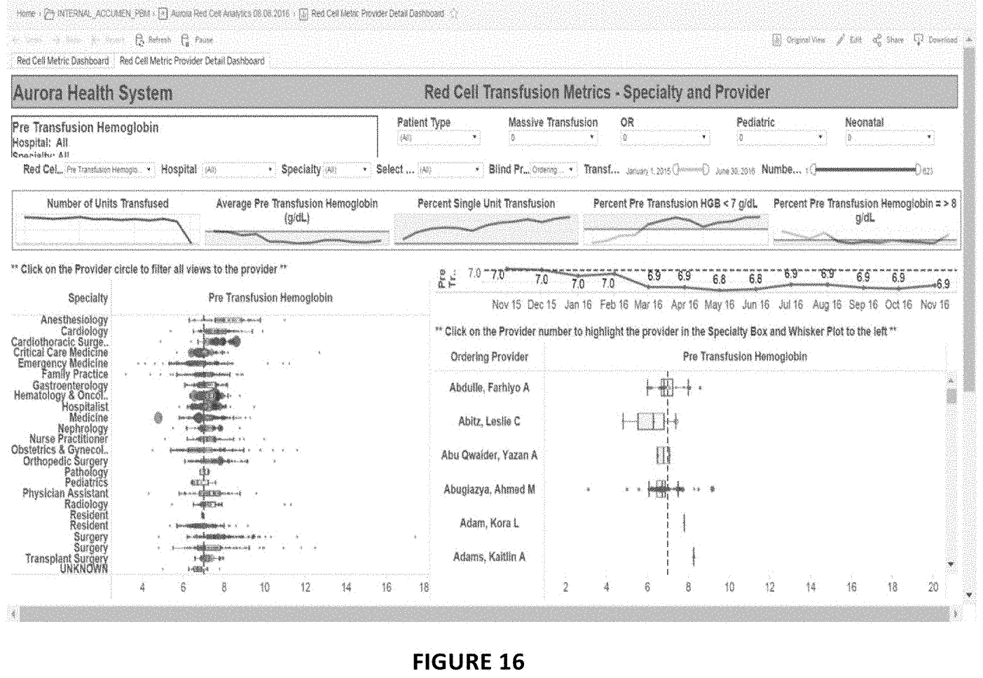

FIG. 15 illustrates an example of a red cell metric dashboard generated by the system. Using the data from the data sources for a particular laboratory, the system may generate this dashboard that displays various generated metrics for red blood cells so that the particular laboratory/facility may improve the underlying patient blood management dimensions shown in FIG. 14. FIG. 16 illustrates an example of a red cell metric provider detail dashboard generated by the system so that the particular laboratory/facility may improve the underlying patient blood management dimensions shown in FIG. 14. In FIG. 16, the statistics/data for each blood provider to the particular laboratory/facility/hospital is displayed based on the data from these blood providers that was ingested into the system.

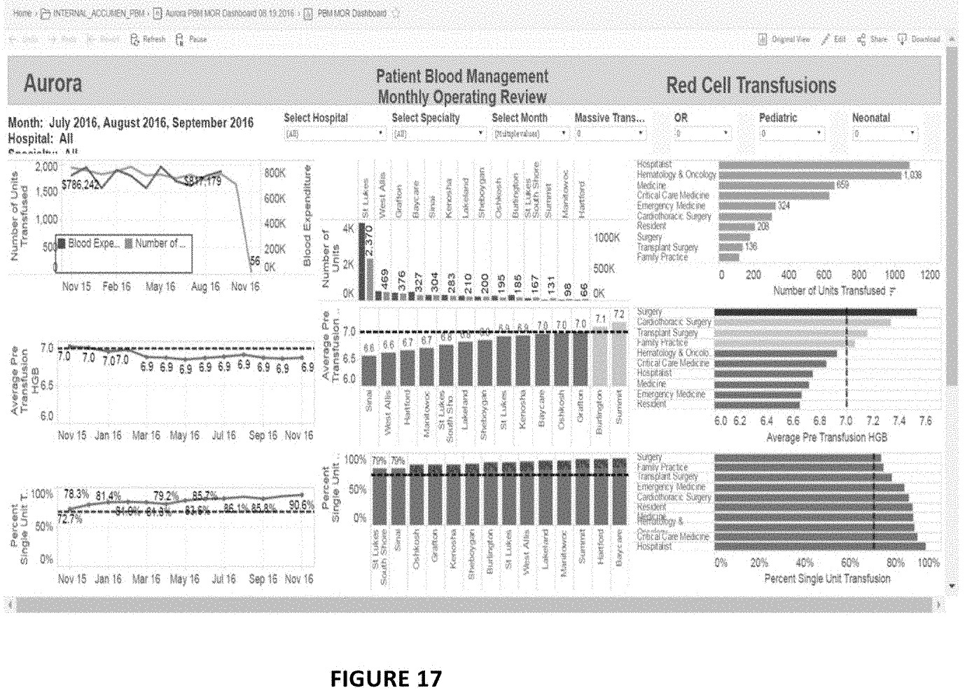

FIG. 17 illustrates an example of a patient blood management monthly operating review screen generated by the system, The screen displays, based on the data from the various data sources, blood data for a particular facility/hospital/lab. As seen in FIG. 17, the analytics may be bar graphs, charts, etc. to more fully illustrate the blood data for the particular facility/hospital/lab.

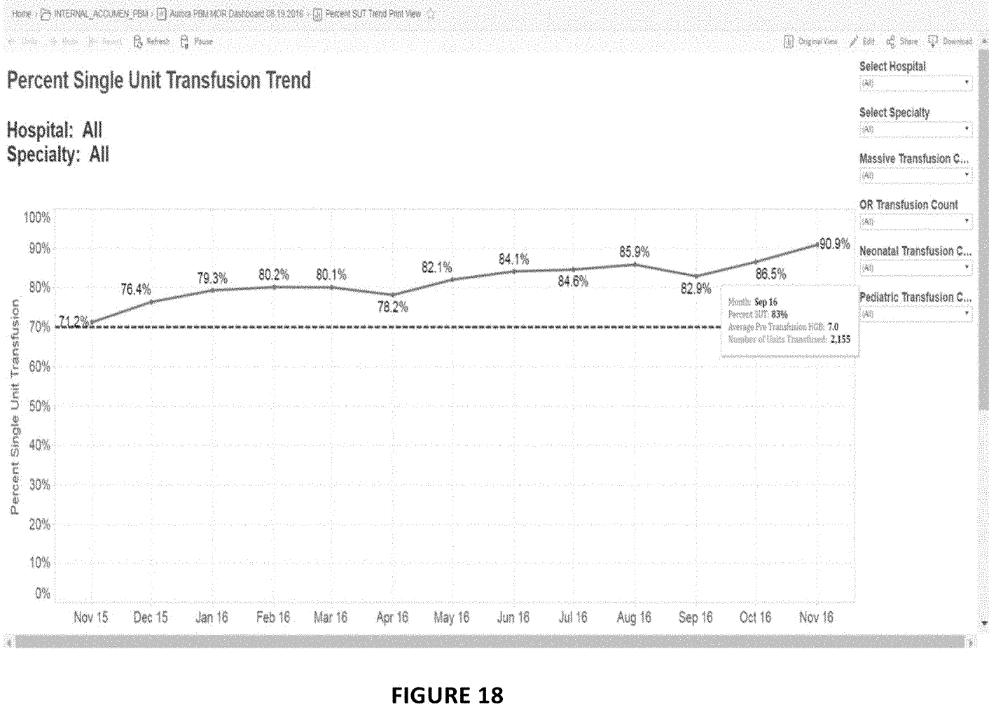

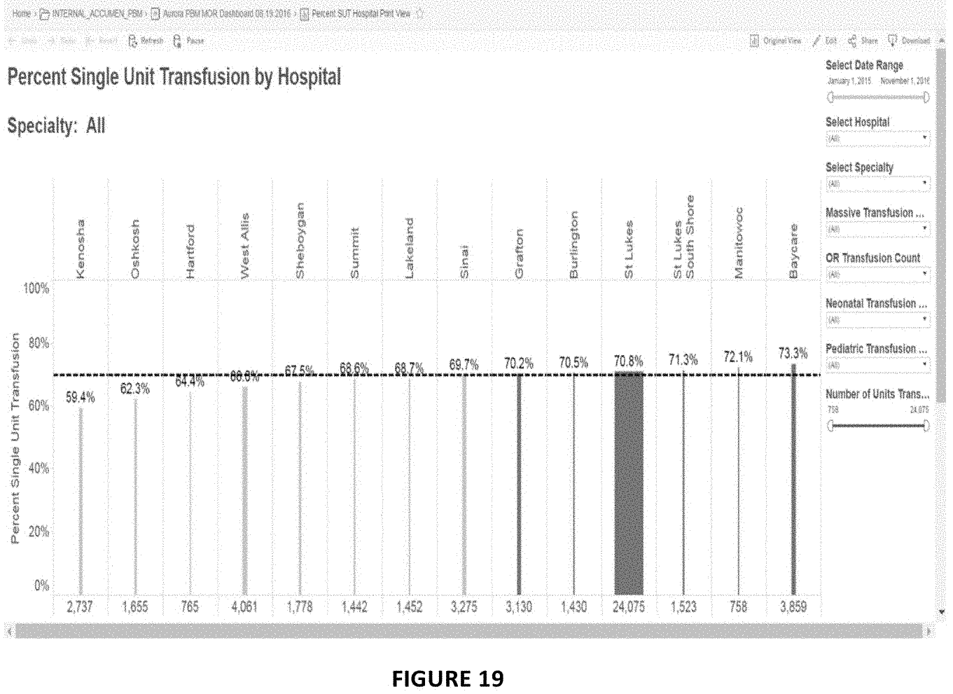

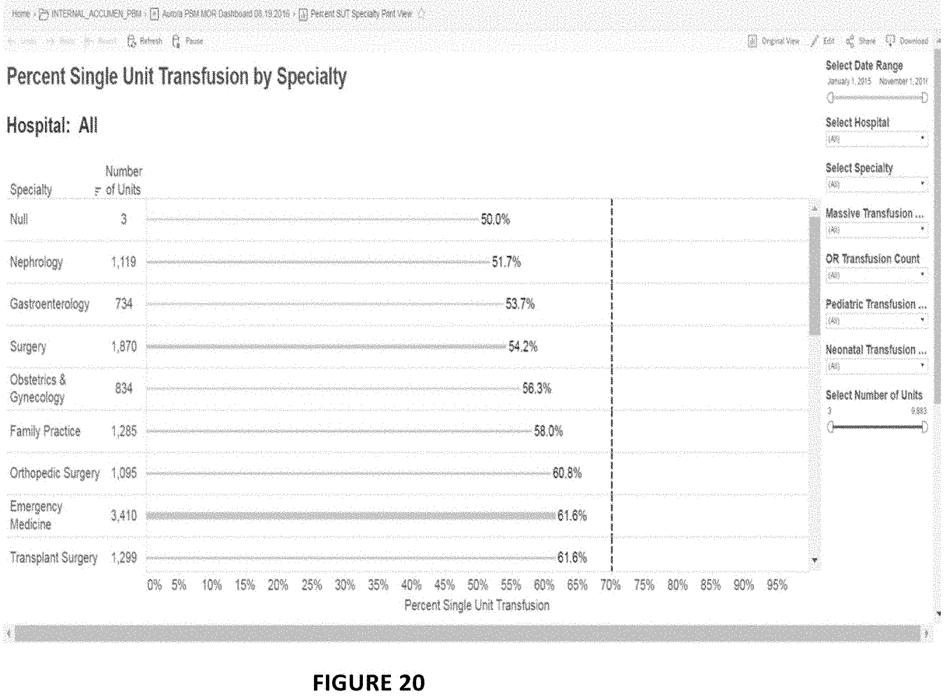

FIG. 18 illustrates an example of a patient blood management percent single unit transfusion trend view generated by the system. The view, using the historical data about transfusions for the particular facility/hospital/lab, may generate a trend analytic over time. In this view, the data is for all hospitals of an entity and all specialties of the entity. FIG. 19 illustrates an example of a patient blood management percent single unit transfusion hospital print view generated by the system. In this view, the same data as shown in FIG. 18 is divided by each hospital of the entity (a company/organization, etc. that runs/owns one or more facilities that may be hospitals, labs, etc. and may have doctors in one or more different specialties) so that the differences between the hospitals may be quickly assessed. Similarly, FIG. 20 illustrates an example of a patient blood management percent single unit transfusion specialty print view generated by the system in which the single unit transfusion is divided up by specialty for all hospitals.

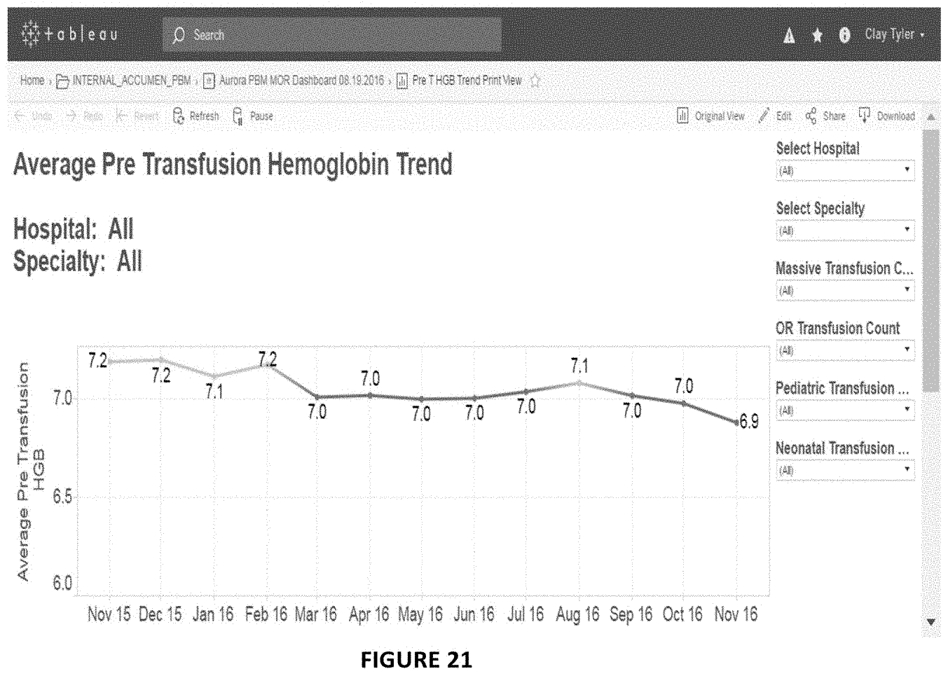

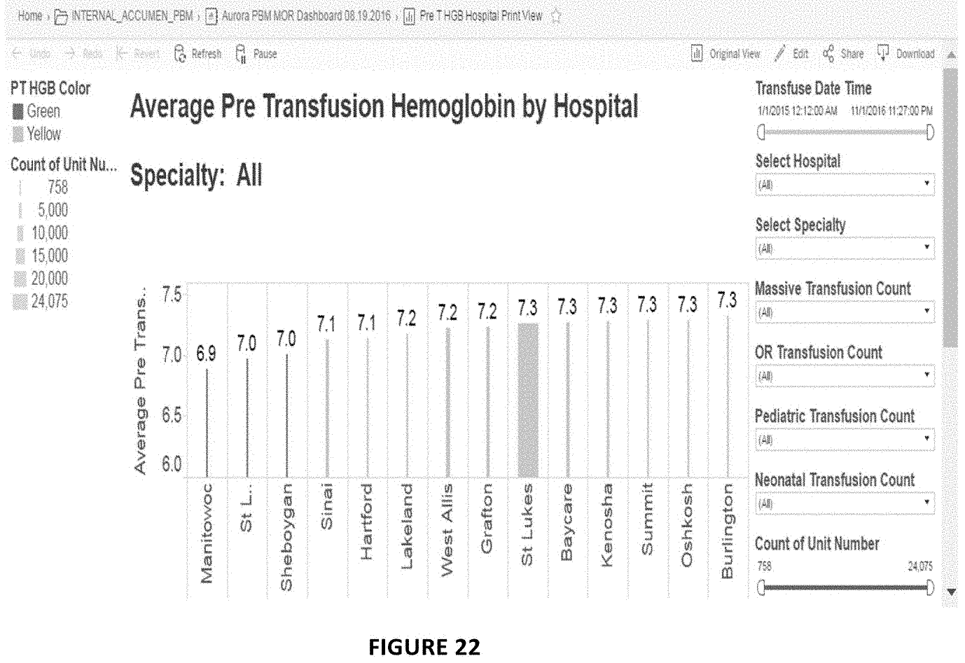

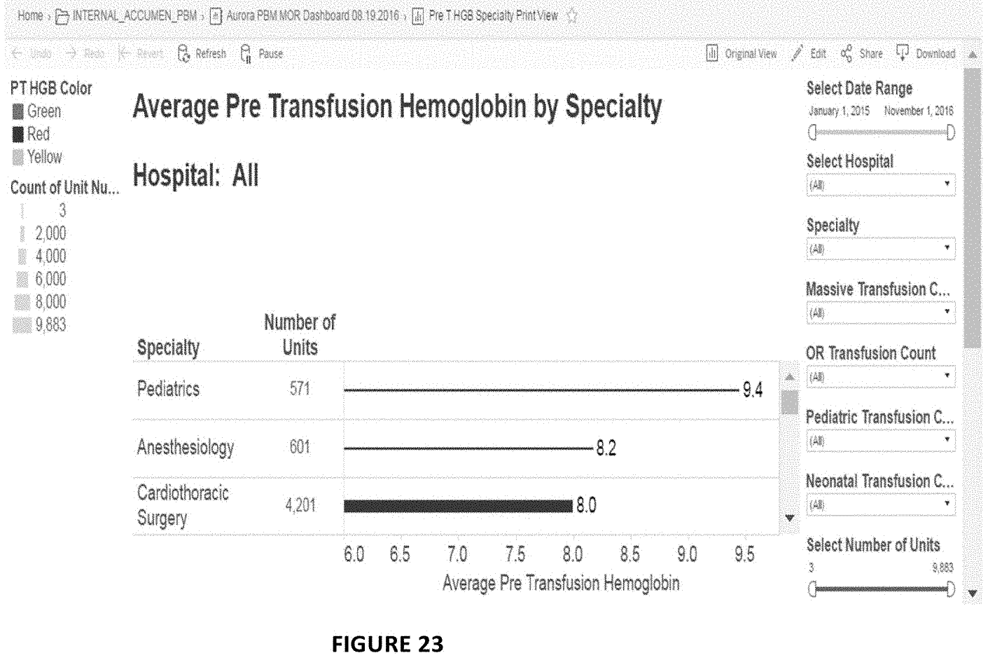

FIG. 21 illustrates an example of a patient blood management pre-transfusion trend print view generated by the system. This view combines the hemoglobin data for all hospitals and all specialties for the entity. FIG. 22 illustrates an example of a patient blood management pre-transfusion hemoglobin hospital print view generated by the system in which the hemoglobin data for the entity is divided by hospital and FIG. 23 illustrates an example of a patient blood management pre-transfusion hemoglobin specialty print view generated by the system in which the hemoglobin data is divided by specialty.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include an/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers; servers or server computing devices such as routing/connectivity components; hand-held or laptop devices; multiprocessor systems; microprocessor-based systems; set top boxes; consumer electronic devices; network PCs; other existing computer platforms; and distributed computing environments that include one or more of the above systems or devices.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Additionally, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise”, “comprising”, and the like are to be construed in an inclusive sense as opposed to an exclusive or exhaustive sense; that is to say, in a sense of “including, but not limited to.” Words using the singular or plural number also include the plural or singular number respectively. Additionally, the words “herein”, “hereunder”, “above”, “below”, and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

1. A method, comprising:
receiving healthcare data from a plurality of data sources wherein each data source has a data format;
conforming the healthcare data from the plurality of data sources to an internal healthcare data format;
providing a set of healthcare data metric rules, each rule associated with a particular healthcare data metric and having a formula for determining the particular healthcare data metric based on the conformed healthcare data and a target for the particular healthcare data metric;
calculating, using the conformed healthcare data, each of a plurality of healthcare data metrics based on the rule for each healthcare data metric; and
generating, using the plurality of healthcare data metrics, a plurality of dashboards wherein each dashboard displays a particular one of the calculated healthcare data metrics in a color that indicates whether the target for the particular one of the calculated healthcare data metric set forth in the rule was met and each dashboard is active and permits a drill down of the conformed healthcare data that is used to generate the dashboard.

2. The method of claim 1, wherein the healthcare data metric further comprises a laboratory performance data metric based on the confirmed laboratory data and a target for the particular laboratory performance data metric and wherein calculating each healthcare data metric further comprises calculating, using the conformed laboratory data, each of a plurality of laboratory performance data metrics based on the rule for each laboratory performance data metric.

3. The method of claim 2 further comprising performing analysis of the laboratory data using the dashboard.

4. The method of claim 1 further comprising setting a global filter, wherein each global filter changes a date range for each of the dashboards generated.

5. The method of claim 4, wherein setting a global filter further comprising selecting one of a plurality of global filters wherein each global filter has a preset date range.

6. The method of claim 1 further comprising storing the conformed healthcare data into a database.

7. The method of claim 2, wherein the conformed laboratory data is laboratory test data from a plurality of laboratories and wherein each dashboard displays data about the laboratory test data.

8. The method of claim 1, wherein the conformed healthcare data is patient blood data from a plurality of facilities and wherein each dashboard displays data about the patient blood data.

9. The method of claim 1, wherein the conformed healthcare data is imaging data from a plurality of facilities and wherein each dashboard displays data about the imaging data.

10. The method of claim 2, wherein the laboratory performance data metric is one of a high priority turn around time, an in patient morning result, an emergency department turn around time, as soon as possible turn around time and unacceptable specimens.

11. A system, comprising:
a computer system having a processor and a memory that implements a backend system;
the processor executing a plurality of lines of instructions to implement an acquisition module that receives laboratory data from a plurality of data sources wherein each data source has a data format;
the processor executing a plurality of lines of instructions to implement a conform module that conforms the laboratory data from the plurality of data sources to an internal laboratory data format;
the processor executing a plurality of lines of instructions to provide a set of healthcare data metric rules, each rule associated with a particular healthcare data metric and having a formula for determining the particular healthcare data metric based on the conformed healthcare data and a target for the particular healthcare data metric;
the processor executing a plurality of lines of instructions to provide an analytics module that calculates, using the conformed healthcare data, each of a plurality of healthcare data metrics based on the rule for each healthcare data metric; and
the processor executing a plurality of lines of instructions to implement a dashboard module that generates, using the plurality of healthcare data metrics, a plurality of dashboards wherein each dashboard displays a particular one of the calculated healthcare data metrics in a color that indicates whether the target for the particular one of the calculated healthcare data metric set forth in the rule was met and each dashboard is active and permits a drill down of the conformed healthcare data that is used to generate the dashboard.

12. The system of claim 11, wherein the healthcare data metric further comprises a laboratory performance data metric based on the confirmed laboratory data and a target for the particular laboratory performance data metric and wherein the processor is further configured to calculate, using the conformed laboratory data, each of a plurality of laboratory performance data metrics based on the rule for each laboratory performance data metric.

13. The system of claim 12, wherein the processor is further configured to perform analysis of the laboratory data using the dashboard.

14. The system of claim 11, wherein the processor is further configured to set a global filter, wherein each global filter changes a date range for each of the dashboards generated.

15. The system of claim 14, wherein the processor is further configured to select one of a plurality of global filters wherein each global filter has a preset date range.

16. The system of claim 11 further comprising storing the conformed healthcare data into a database.

17. The system of claim 12, wherein the conformed laboratory data is laboratory test data from a plurality of laboratories and wherein each dashboard displays data about the laboratory test data.

18. The system of claim 11, wherein the conformed healthcare data is patient blood data from a plurality of facilities and wherein each dashboard displays data about the patient blood data.

19. The system of claim 11, wherein the conformed healthcare data is imaging data from a plurality of facilities and wherein each dashboard displays data about the imaging data.

20. The system of claim 12, wherein the laboratory performance data metric is one of a high priority turn around time, an in patient morning result, an emergency department turn around time, as soon as possible turn around time and unacceptable specimens.
